(19) United States
(12) Patent Application Publication
(10) Pub. No.: US 2024/0112756 A1
(43) Pub. Date: Apr. 4, 2024

(54) METHOD FOR ANALYZING GENETIC ELEMENTS AND SURROUNDINGS

(71) Applicant: The Charles Stark DraperLaboratory, Inc., Cambridge, MA (US)

(72) Inventor: Laura Seaman, Cambridge, MA (US)

(21) Appl. No.: 18/360,957

(22) Filed: Jul. 28, 2023

Related U.S. Application Data
(60) Provisional application No. 63/377,773, filed on Sep. 30, 2022.

(51) Int. Cl. G16B 30/20

(52) U.S. Cl. CPC G16B 30/20

(57) ABSTRACT

A system and processes enable determination of the genetic location of a sequence of interest including markers of genetic engineering. More specifically, a system and/or a series of processes are used independently or together to allow assembly, annotation, and visualization of genetic elements of interest (GEI) as well as their genetic surroundings from sequencing data. This can enable users to quickly and efficiently review and interpret the results so they can understand what GEIs are present in a sample, where they are, and their surroundings. It can be used to enrich sequences of interest or with sequences that have not been enriched and can help answer research questions such as understanding the on and off target effects of genetic engineering or discovery of novel gene homologs.

### RELATED APPLICATIONS

This application claims the benefit under 35 USC 119(e) of U.S. Provisional Application No. 63/377,773, filed on Sep. 30, 2022, which is incorporated herein by reference in its entirety.

### GOVERNMENT SUPPORT

This invention was made with Government support under contract number N66001-18-C-4503, awarded by the U.S. Department of Defense. The Government has certain rights in the invention.

### BACKGROUND OF THE INVENTION

Probe-based sequencing is a technique used to find the genetic elements of interest (GEI) such as sequence of nucleotides. The specific methods can vary, but generally involve several steps. The GEI or specific segment of DNA to be sequenced is identified. This could be a gene known to be associated with a specific disease, for example, or it could be a region of the genome that has not been well-studied or could be an RNA sequence of a virus in waste water. Once the GEI has been identified, it is usually amplified using a method like Polymerase Chain Reaction (PCR). This produces a large number of identical copies of the target sequence to be sequenced. A probe is often a short piece of single-stranded DNA or RNA that is complementary to the GEI. The probes are usually affixed to a substrate or labeled such as with a fluorescent dye, so that they can be detected. The probes are then mixed with the sample possibly containing the GEI under conditions that allow the probe to bind to its complementary sequence in the target in a process known as hybridization. After hybridization, the excess probe that did not bind is washed away. This leaves behind only the GEI that has been bound by the probe. Finally, the probe-bound target is then detected. This is typically done using a fluorescent detector that identifies the labeled probes. Because the sequence of the probe is known, and the probe is complementary to the target sequence, this effectively reveals the GEI.

In addition to this basic method, there are many variations and enhancements that can be used to improve accuracy, increase throughput, or sequence longer stretches of DNA or RNA. Some methods, for example, use a series of probes that are each complementary to a different sub-sequence of the target, allowing for the sequencing of much longer stretches. Other methods use sophisticated computational algorithms to improve accuracy or to infer the sequence from incomplete or noisy data.

### SUMMARY OF THE INVENTION

The present invention can allow users to determine the genetic location of a sequence of interest including markers of genetic engineering. More specifically, a system and/or a series of processes are used independently or together to allow assembly, annotation, and visualization of genetic elements of interest (GEI) as well as their genetic surroundings from sequencing data. This can enable users to quickly and efficiently review and interpret the results so they can understand what GEIs are present in a sample, where they are, and their surroundings. It can be used to enrich sequences of interest or with sequences that have not been enriched and can help answer research questions such as understanding the on and off target effects of genetic engineering or discovery of novel gene homologs.

When performing enrichment, DNA homology and sequencing are used to enrich the GEIs relative to the background sequences. More specifically, probes are employed that are based on portions of the GEI and affixed to a plate or bead. Sample DNA is then mixed with the probes which capture sequences that match the probes while others can be separated out. The process is then used to assemble sequences showing which GEI were in the samples and their surroundings. Unlike traditional assembly, the present processes typically do not assemble the whole genome of the sample DNA, instead the processes focus on just the GEI and their immediate surroundings (up to 10s of kilobases (kb). Because the sequences of interest are overrepresented in enriched data, traditional assembly algorithms do not perform well since the assumption of a consistent read depth is violated. To overcome this, the process includes a series of bioinformatic tools with subprocesses to produce the desired information and outputs.

In general, according to one aspect, the invention features a system comprising a DNA sequencer that generates a series of reads associated with a sample containing DNA and a computing device that analyzes the reads to assemble, annotate, and/or visualize genetic elements of interest.

In embodiments, the DNA sequencer is used in combination with probe based enrichment.

The computing device can employ smart-filtering to select reads for assembly that will optimize assembly output regardless of the number or distribution of reads.

The reads can be selected based on how much information they contain about the genetic elements of interest and its flanking region such that the reads might be broken into the three types: fully mapped, half mapped, and unmapped reads. In addition, the computing device can perform assembly using many half-mapped reads, a smaller number of fully mapped reads, and the smallest number of unmapped reads.

Preferably, the computing device employs an assembly quality analysis to determine which node sets are to be used and/or a smart selector.

An assembly quality score is useful to compare different runs of the same sample in the sequencer to score and rank for each run. The runs can be compared using the average ranking, and the run with the lowest average ranking was set as the default for the pipeline.

In some implementations, the computing device employs a crosstalk metric created to predict whether a GEI exists in a sample from crosstalk or not.

Also, the system might be a laboratory instrument or a field instrument to analyze waste water, for example.

In general, according to another aspect, the invention features a method of analysis of a DNA-containing sample. The method comprises generating a series of reads associated with a sample containing DNA using a sequencer and analyzing the reads to assemble, annotate, and/or visualize genetic elements of interest.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1A is a block diagram of a system to which the present invention is applied;

FIG. 1B is a process diagram showing operations executed by an analysis app;

FIG. 2 is a process diagram that expands upon the seven main steps provided in FIG. 1B;

FIG. 3 is a schematic diagram illustrating the DNA from the sample;

FIG. 4 is a screenshot of a graphic user interface displayed on the display 84 showing an assembled region;

FIG. 5 is a flow diagram showing the operation of the Smart-Selector;

FIG. 6 is a schematic diagram showing the components in the assembly quality score algorithm;

FIG. 7 is plot of showing an example of a read depth pattern marked as incorrect merging;

FIG. 8 is a screenshot of the graphic user interface displayed on the display 84 illustrating a node in which the same genetic elements were found repeated as can be seen by the same read depth and Blast hits;

FIG. 9 is a flow diagram showing how each round of filtering uses the node set from the last round of filtering as input;

FIG. 10 is a flow diagram illustrating the BLAST optimization algorithm;

FIG. 11 illustrates a notation scheme;

FIGS. 12A, 12B, 13A, 13B, and 14 are screenshots of the graphic user interface displayed on the display 84; and

FIG. 15 is a schematic diagram depicting questions asked by experts that have been quantified by the metric.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

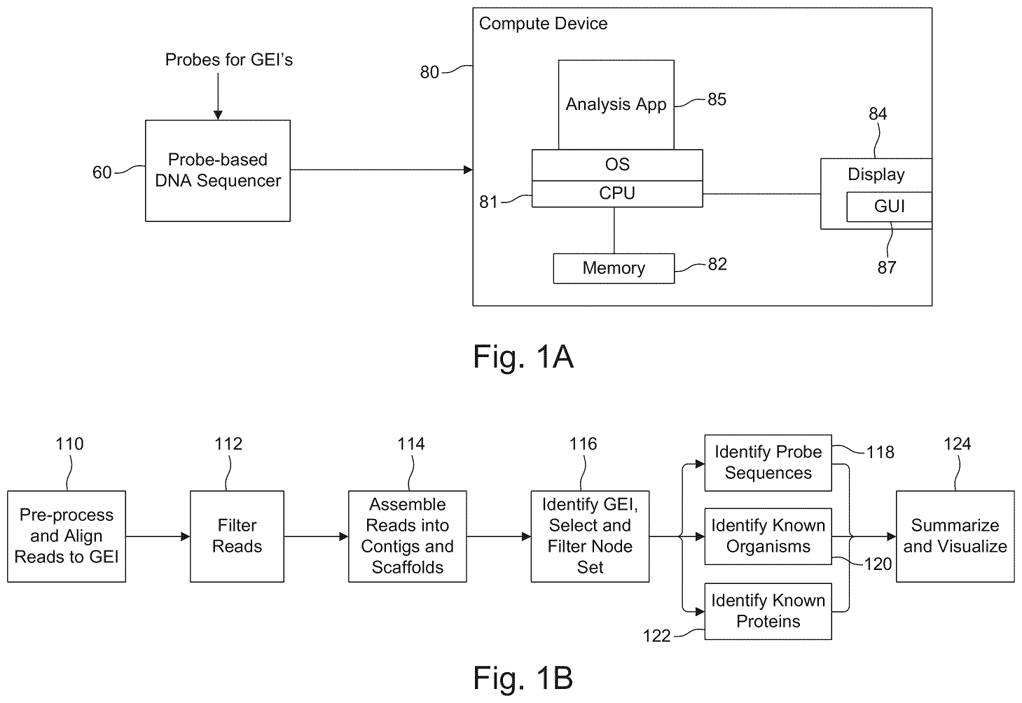

FIG. 1A shows the system to which the present invention is applied. In general, this can be a laboratory system or a portable device that is used in the field to analyze novel DNA sequences. One specific example is to analyze viruses found in wastewater at a wastewater treatment facility.

A DNA sequencer 60, such as an illumina sequencer, analyzes a sample containing DNA strands. The resulting sequencing data and specifically a series of reads, such as in the form of read files in FASTQ format, are provided to a computing device 80.

The computing device 80 could be a desktop computer, server or server cluster. It includes a processor or central processing unit (CPU) 81, memory 82, and a display 84. Executing on the processor 81 is an operating system and an analysis app 85. The app 85 receives user configuration and the sequencing data and processes that information to assemble, annotate, and visualize genetic elements of interest (GEI) as well as their genetic surroundings from sequencing data. This information is then presented on the display 84 in a graphical user interface 87 rendered on that display.

FIG. 1B is a process diagram showing six main operations executed by the analysis app 85 executing on the computer device 80.

The process takes in sequencing data and specifically a series of reads. First, the adaptors are trimmed off the reads. These trimmed reads are then aligned to the GEI in step 110. Second, smart-filtering is performed to select reads for assembly that will optimize assembly output regardless of the number or distribution of reads in step 112. Third, the filtered reads are assembled into contigs and scaffolds in step 114. Next, it is determined which nodes contain GEI in step 116. For this, Basic Local Alignment Search Tool (BLAST) can be employed. A custom assembly quality algorithm is then preferably applied to determine which node set is used for the remainder of the pipeline, although this step could be skipped at the possible expense of a longer run time. Once selected, the nodes are fed through a series of annotation steps 118, 120, 122 to identify the probe sequences, organisms, and known proteins and thus genes contained in each node. Finally, the app summarizes this information into tables and two main sets of visualizations (static figures provide a fast, high-level overview and dynamic figures provide much more detail). This process can be implemented in the Python programming language using Snakemake (odsc.com/speakers/snakemake-a-python-pipeline-toolbox/) to connect the combination of Python scripts and bash commands using a series of scalable and repeatable rules. Snakemake is a workflow management system tool to create reproducible and scalable data analyses. Workflows are described via a human readable, Python based language. They can be seamlessly scaled to server, cluster, grid and cloud environments, without the need to modify the workflow definition. Finally, Snakemake workflows can entail a description of required software, which will be automatically deployed to any execution environment.

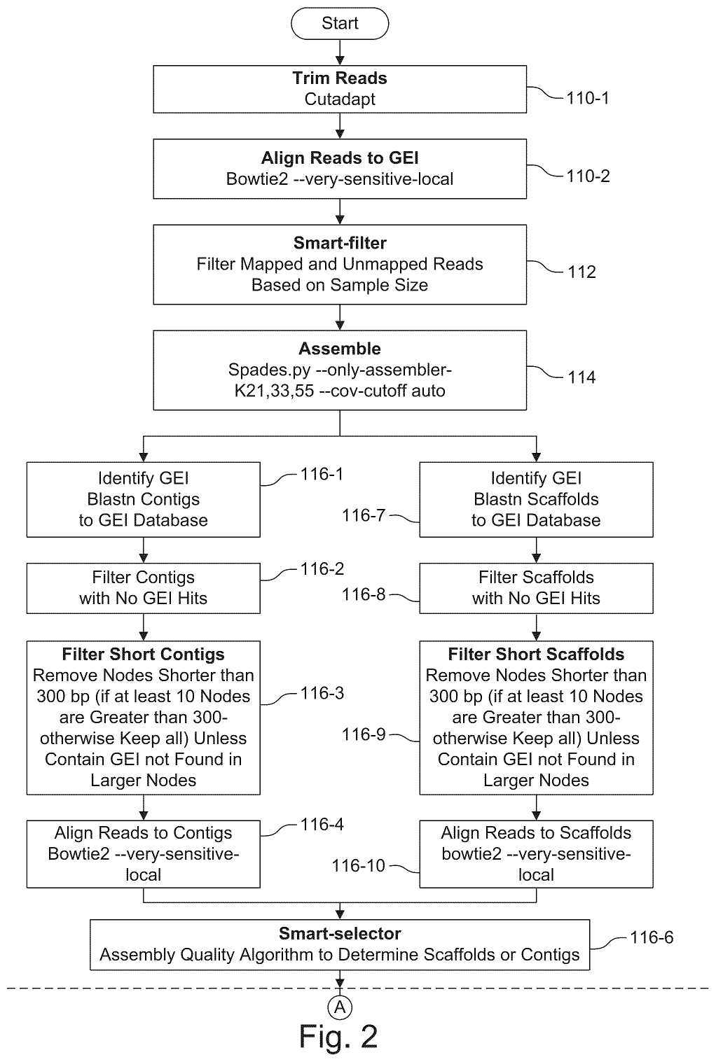

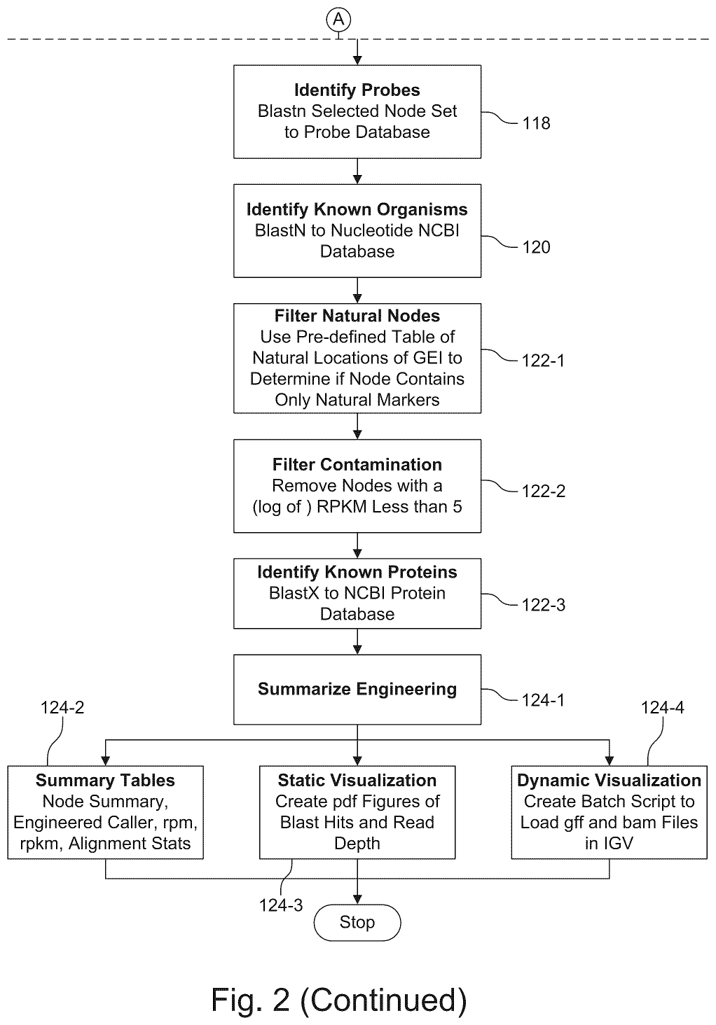

FIG. 2 is a process diagram that expands upon the seven main steps provided in FIG. 1B and provides a lower-level overview of the developed system.

The inputs to compute device 80 for the pipeline of the analysis app 85 are pairs of read files in FASTQ format from the sequencer 60. One file contains the forward reads and the second file contains the reversed read for every read pair. The reads can be paired-end reads which generally are 300-500 basepairs (bp) apart, or mate-pair reads which generally are 3000-5000 bp apart and therefor provide improved information on the regions flanking GEI. Cutadapt, a bioinformatics tool for cleaning data, is used to find and remove adapter sequences found in the incoming reads (Martin, M. (2011). Cutadapt removes adapter sequences from high-throughput sequencing reads. EMBnet. journal, 17(1), 10-12. The adapter sequences are pre-defined and given as inputs to the cutadapt software. Preferably, the “-m 20” argument is included to discard read pairs with at least one read shorter than 20 base pairs.

After trimming in step 110-1, the reads are aligned to the GEI using bowtie2 (see Langmead, B., &. Salzberg, S. L. (2012), Fast gapped-read alignment with Bowtie 2. Nature methods, 9(4), 357, as shown in step 110-2. The purpose of the alignment is to identify which reads contain information about the GEI. This information is used for filtering reads later in the pipeline. Prior to running the pipeline, a FASTA file with the nucleotide sequence of the GEI was compiled and converted into a bowtie2 index. During the pipeline, the trimmed reads and a bowtie2 index of the target genome (GEI) are provided as inputs to the alignment algorithm along with specific parameters needed for mate pair or paired end read alignment (insertion size and expected read orientation). The output of the alignment is sequence alignment/map (SAM) file, which is sorted into a binary alignment/map (BAM) file. The BAM file contains information about each read, such as if it is mapped or unmapped. A mapped/aligned read means its nucleotide base pair sequence is sufficiently similar to a region of the GEI.

The goal of the smart-filter in step 112 is to extract a set of reads that will lead to the best possible performance of the assembly algorithm. Generally, inputting all the reads for a given sample to assembly overwhelms the assembler with irrelevant and difficult-to-assemble information causing assembly to timeout or produce poorly assembled nodes, such as hundreds of small pieces for a single GEI. Usually, assembly algorithms are developed with an assumption of even read depth (a consistent number of reads at all points in the genome). Since this system is developed to focus on specific elements within a genome and uses sequence enrichment to make the GEI overrepresented in the sequencing data relative to shot gun sequencing, the read depth profile of our datasets violates that assumption thus requiring the smart-filter.

It should be noted that in general, this disclosed pipeline can work even without the laboratory-based enrichment.

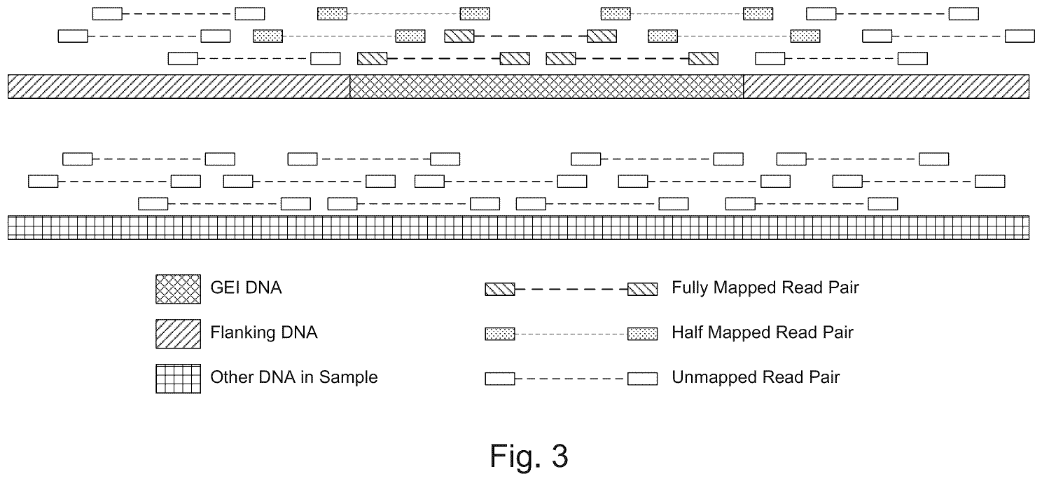

FIG. 3 illustrates the reads. When looking for GEI, there are three types of reads. In fully mapped reads (orange), both reads in the pair align to the GEI. In half mapped reads, one end aligns to the GEI while the other does not, providing valuable information on the flanking region. In unmapped reads, neither end aligns to the GEI.

The developed smart filter selects different numbers of reads depending on how much information they contain about the GEI and its flanking region. The reads for a sample are broken into the three types: fully mapped, half mapped, and unmapped reads. The different types of reads contain different types and amounts of information and thus are treated differently when filtering. A fully mapped read is defined as a read pair in which both of the reads in the pair align to a GEI or reference genome. Fully mapped reads provide a lot of information about the specific sequences of interest but in the case where those segments were enriched, they can be overrepresented in the results and keeping a subset can improve performance. The second read type is half-mapped reads which are those in a read pair in which exactly one read aligns while its partner read in the pair does not. Half mapped reads are generally the most valuable as they provide information on both the GEI and their surroundings. These are critical data to successfully assembly the flanking regions. The third read type are fully unmapped reads, which are reads where neither read in the pair mapped. Unmapped reads might contain information on the flanking region but also contain lots of irrelevant information like the sequences of the rest of the organism(s) in the sample.

To increase assembly performance, the smart filter keeps more reads of the types with the highest information content. This means that assembly uses many half-mapped reads, a smaller number of fully mapped reads, and the smallest number of unmapped reads. In addition to keeping the reads with the most valuable information, the smart filter also adjusts the distribution of reads over the GEI so that the selected sequences have a more even read depth profile to match the assembly algorithms assumption. Table 1 shows the full smart-filter algorithm, including the examples of specific thresholds that worked well during testing with experimental data. The parameters implemented in the smart filter were developed by testing various parameter sets on multiple types of samples.

TABLE 1
Reads kept by the Smart-Filter.
The Smart-Filter selects different numbers of reads and uses a
different filtering algorithm depending on the type of read.

| | Small Sample | Large Sample |
|---|---|---|
| Read Type | (<40M reads) | (>40M reads) |
| Unmapped | Keep 100,000 | Keep 100,000 |
| | random reads | random reads |
| Half mapped | Keep 20 random | Remove peaks to |
| | reads per base of GEI | read depth of 4000 |
| Fully mapped | Keep 15 random | Remove peaks to |
| | reads per base of GEI | read depth of 2000 |

The assembly quality algorithm (explained hereinbelow) was used to rank varying parameter sets. Parameter options include any combination of peak removal; down sampling by GEI; keep/remove unmapped reads; keep/remove unpaired reads; remove duplicates, and randomly downsample all reads.

The smart-filter currently differentiates between large and small samples (large=over 40M reads). For large samples, it was determined that peak removal improved assembly results. The peak removal algorithm is used to keep a constant read depth along the GEI to better match the constant read depth expected by assembly.

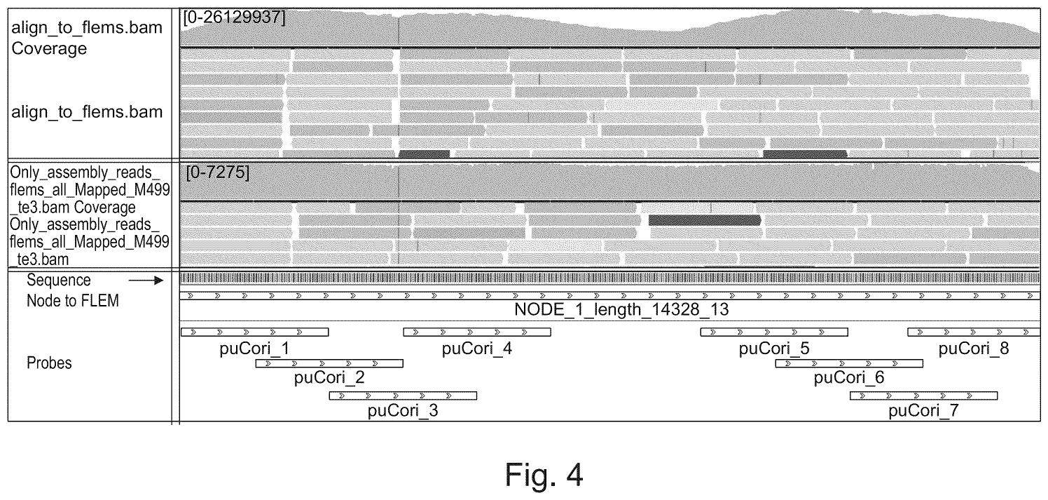

FIG. 4 is a screenshot of a graphic user interface displayed on the display 84 showing an assembled region, showing the read depth over a GEI before and after peak removal. Note that original read depth (top grey histogram) maxes out at 2,612,937 reads and after peak removal (middle grey histogram) the read depth is 0-7252.

FIG. 4 shows the read depth before and after peak removal for an assembled region with one GEI. For small samples, it was determined that downsampling, where a given number of reads based on the length of each GEI were randomly selected, performed best. In both cases keeping a relatively small number of unmapped reads also improved performance (exact numbers and thresholds are in Table 1). In addition to the smart filter, the system, through the config file, allows each read type (fully mapped, half mapped, fully unmapped) to be included entirely, excluded or have some filtering performed. When using the smart-filter or any other option, the selected paired reads are then all merged into a single FASTQ file prior to assembly. By using FASTQ, the system preserves the quality information about the reads increasing the accuracy of assembly. Unpaired reads, created during the trimming step, are merged into a separate FASTQ file if selected, although they are not used by default.

The assembly step creates the sequences of DNA that will ultimately be visualized and used to classify each sample as engineered, or not engineered. The input to assembly has been optimized by the smart-filter and the output is then passed through the smart-select algorithm to determine which results to annotate and continue analyzing. After the reads are filtered by the smart-filter, they are sent through the SPAdes assembler (Bankevic Nurk, S., Antipov, D., Gurevich, A. A., Dvorkin, M., Kulikov, A. S., . . . & Pevzner, P. A. (2012). SPAdes: a new genome assembly algorithm and its applications to single-cell sequencing. Journal of computational biology, 19(5), 455-477) as shown in step 114 of FIG. 2. The assembler produces contigs, which are continuously assembled stretches of nucleotides, as well as scaffolds, which can have some unknown nucleotides in the sequence by using paired reads to determine the longer connections. Scaffolds can provide relative positions of multiple GEI and larger flanking regions although sometimes produce incorrect concatenation of contigs.

Occasionally, SPAdes fails to assemble nodes based on a lack of input reads. This occurs when one sample does not have any GEI and thus very few reads align to a GEI. If a sample has very few reads (currently set to 10,000) going into assembly and assembly fails to produce nodes, the sample is classified as not containing any GEI and no further steps are performed.

The assembler produces both contigs, which are continuously assembled stretches of nucleotides, and scaffolds, which can have some unknown nucleotides in the sequence by using paired reads to determine the longer connections. The system must determine on a per-sample basis which node set should be used for the remainder of the pipeline. To do this, steps are performed on both node sets and the results are compared using the assembly quality algorithm.

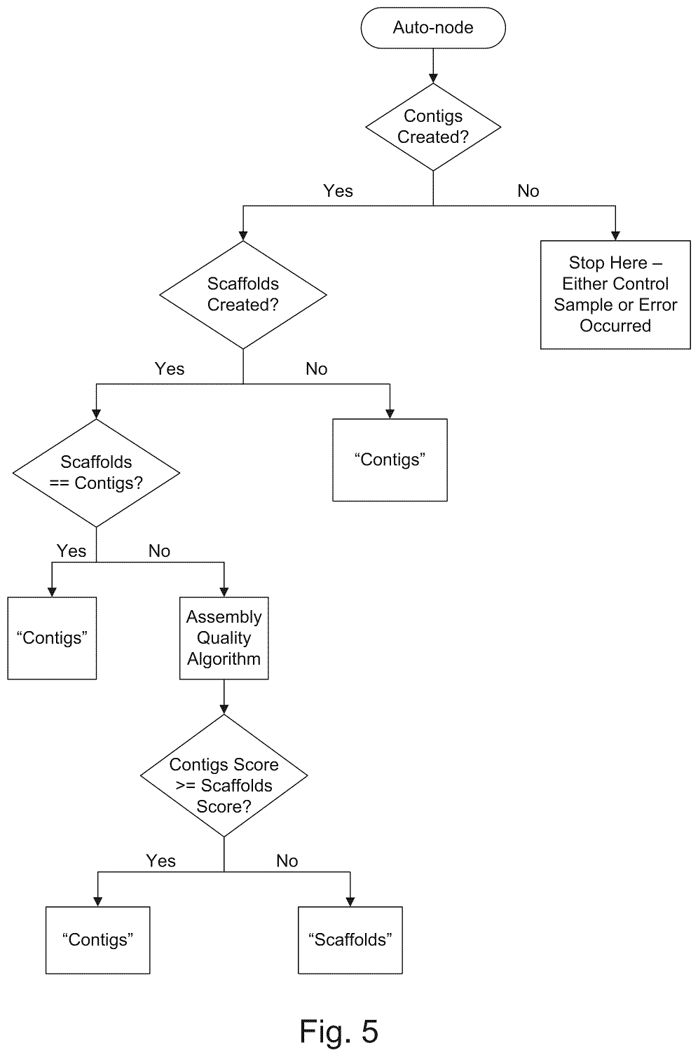

FIG. 5 shows the operation of the Smart-Selector.

The first step performed is to use BLAST to compare the assembled nodes to the database of GEI to identify which GEI were assembled in each sample as shown in steps 116-1 and 116-7 of FIG. 2. This will provide annotations for the nodes that can be used for the first stage of filtering. Any node that does not have at least one BLAST hit to GEI is discarded as shown in steps 116-2 and 116-8 of FIG. 2. If a sample does not have any nodes containing a GEI, the sample is classified as a not engineered and no further steps are performed. Short nodes are then discarded based on length and number of nodes generated for each node set as shown in steps 116-3 and 116-9 of FIG. 2. Specifically, if there are 10 or fewer nodes at least 300 base pairs long, no nodes are filtered. Otherwise, any node shorter than 300 base pairs that does not include a BLAST hit to any GEI is discarded.

Next, the trimmed reads from the pre-processing stage of the pipeline are aligned to the filtered nodes as shown in steps 116-4 and 116-10 of FIG. 2. The alignment results are used in the assembly quality algorithm, and during visualization in the final stage of the pipeline to provide a qualitative reflection of confidence. A high and consistent read depth yields higher confidence that the node accurately depicts the contents of the sample. Occasionally, the scaffolds file will be empty or contain the same sequences as the contigs file. In this case, the above steps are only performed on contigs and contigs are automatically chosen as the node set to be used for the remainder of the pipeline.

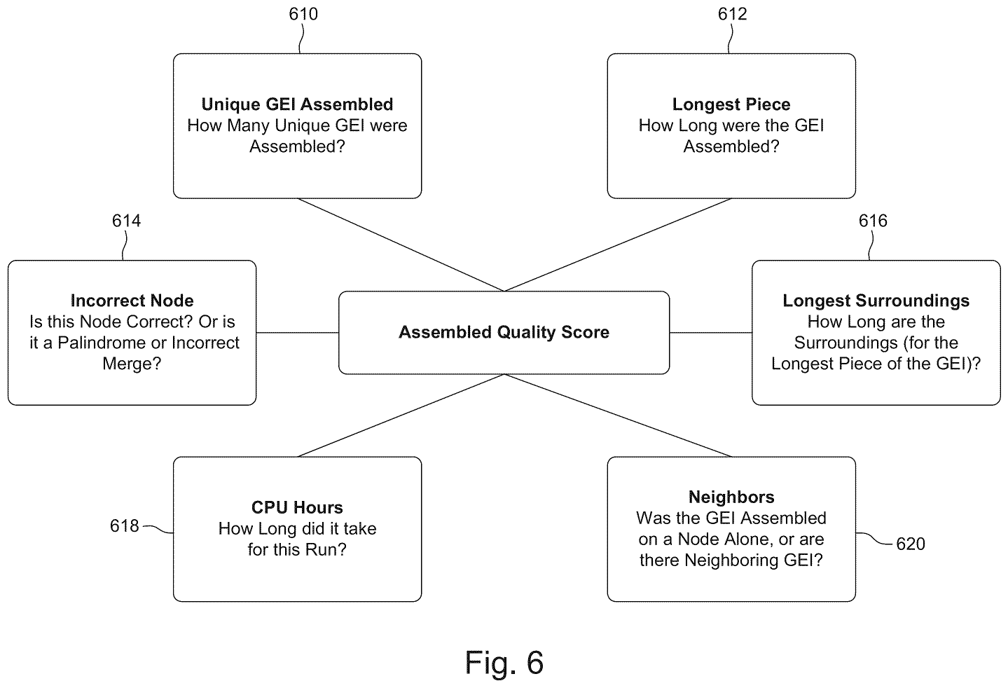

FIG. 6 shows the components in the assembly quality score algorithm. It includes several factors such as how many unique GEIs were assembled 610, how long the assembled GEIs were 612, whether a node was incorrect 614, how long the surroundings were for the longest piece of the GEIs 616, how much CPU time was required for the analysis 618, and were the GEIs assembled on a node alone or were there neighboring GEIs 620.

The assembly quality algorithm is used to compare different “runs” of the same sample. Here, a run refers to a unique set of parameters used by the pipeline that produce a set of nodes (either contigs or scaffolds, assembled through SPAdes). The assembly quality algorithm is implemented in the pipeline as shown in step 116-6 of FIG. 2. It has also been used for hyper-parameter tuning of the pipeline to determine which parameters performed best. During each run of the pipeline and each sample, either scaffolds or contigs must be selected and sued for the final outputs and visualizations. The assembly quality algorithm is used to compare contigs to scaffolds. The node set scoring higher is used for the remainder of the pipeline.

The system assesses each run of the pipeline for a given sample on various metrics, giving a score and rank for each run. When being used in the smart-selector in the pipeline, there is only one sample and two runs (differing by scaffolds or contigs). In the case of hyper-parameter tuning, approximately a dozen runs on each of 50 samples each were analyzed. The runs were compared using the average ranking, and the run with the lowest average ranking was set as the default for the pipeline. Scores from the algorithm cannot be compared across samples since the scores are dependent on number of GEI in a sample (more GEI means the maximum potential score is higher).

FIG. 6 depicts the various questions that go into scoring a node set. The metrics and specific weightings used are described in more detail below. The weightings of each component were developed using trial and error and manual review to determine how to balance the goal of having large contigs against the need to make sure that genetic elements are not being combined incorrectly.

The most heavily weighted portion of the score refers to number of GEI found 610. A run receives 5 points per unique GEI assembled. A run will receives 2 points per GEI for assembling the longest piece of the GEI 612. To determine this, the longest piece of the GEI in any run is selected, and any run within 5 base pairs or 10% of the longest piece size will get the points. One point is awarded per GEI with a neighbor 620. A neighbor refers to another GEI on the same node. Only the longest piece of each GEI is considered for this metric.

Points are then awarded for having the longest surroundings 616. Only the longest piece of each GEI is considered. A run will receive 0.5 points for each GEI that has the largest surroundings. To determine the largest surroundings, the surroundings of GEI from all runs are considered. “Surroundings” refers to base pairs not covered by a GEI on the node. Any run within 5 base pairs or 10 percent of the largest surroundings receives the points. CPU time is then incorporated into the score 618 as available, mainly for tiebreak purposes. The CPU time for a given sample is subtracted from the CPU time of the sample with the longest time and this value is taken as a fraction over the longest time for a final value between 0 and 1.

Occasionally, assembly produces an incorrect node 614 either by merging two sequences that should not be merged or creating a palindrome node (a node that repeats incorrectly). A node is classified as an incorrect merge if there is a sudden drop in read depth between neighboring base pairs. A sudden drop is defined as at least a 500% change in read depth between adjacent base pairs along with the ratio of average read depth on either side being greater than 50. The average read depth on either side is taken over a window that is 10% the size of the node.

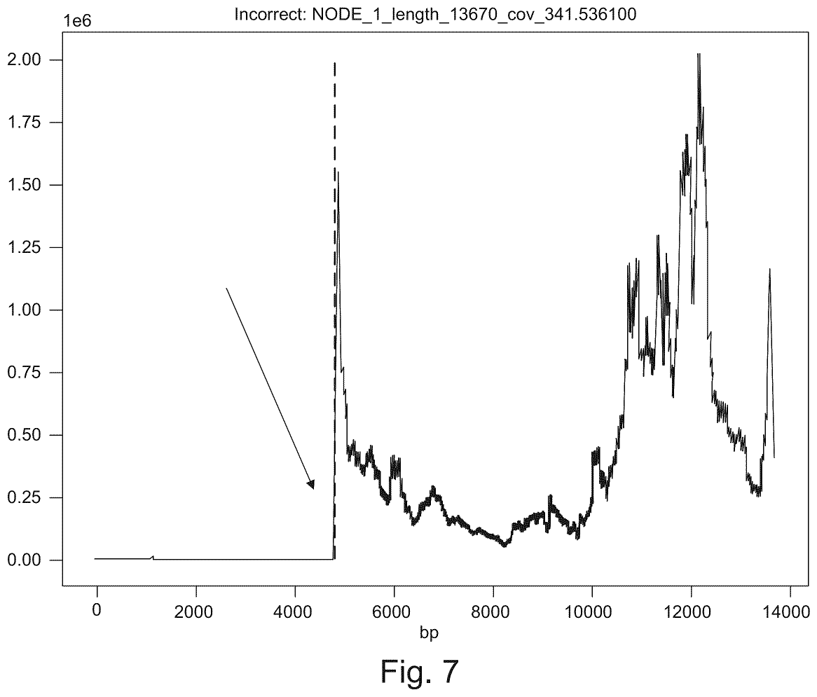

FIG. 7 is an example of a read depth pattern marked as incorrect merging.

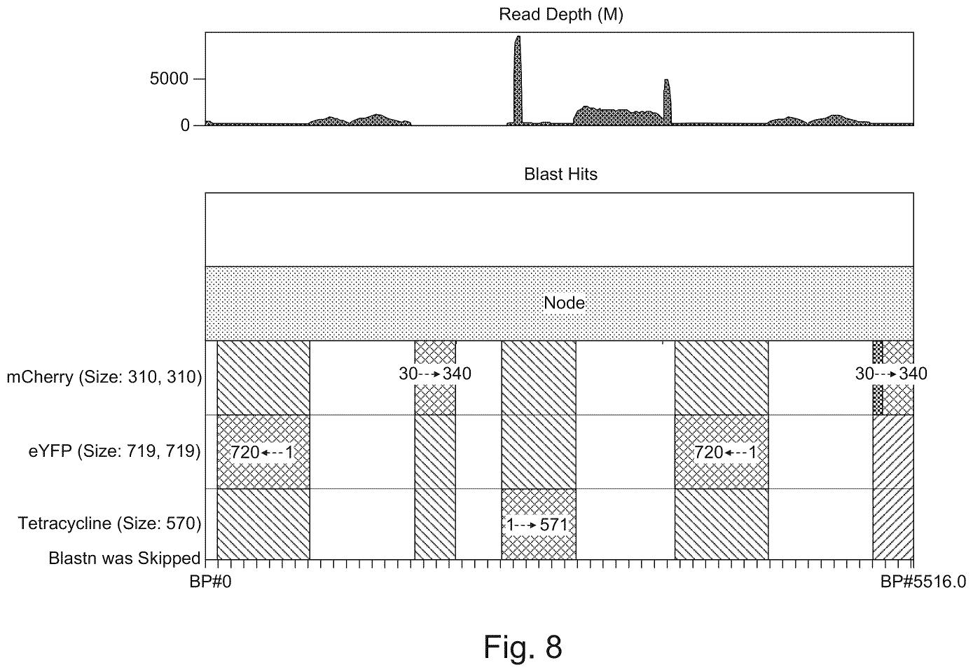

FIG. 8 shows a node in which the same genetic elements were found repeated. A node is classified as a palindrome/repeated if the GEI (except for possibly a center GEI) appear twice in the annotations, with the same portion of each GEI mapped both times.

The assembly quality algorithm searches for either case and makes respective adjustments. Five points are deducted from the overall score for each incorrectly merged node. Palindrome nodes are treated as shorter nodes, excluding the repetitive regions for counting surroundings and other metrics within the smart-scorer.

The node set selected by the assembly quality algorithm is then annotated to identify probe locations, known organisms and known proteins. The locations of GEI were determined as part of the assembly selector algorithm so now the selected contigs are compared to other databases for additional context.

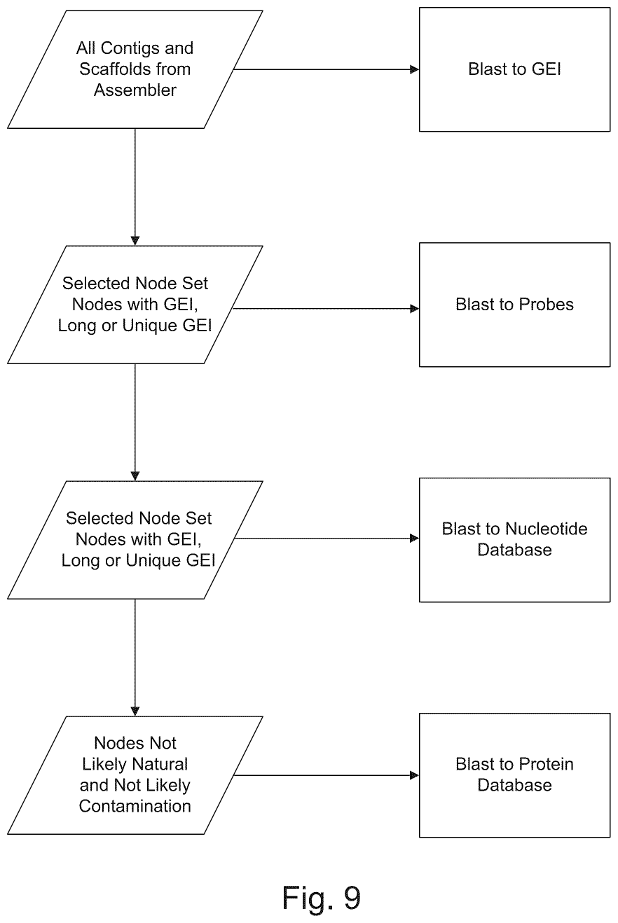

As shown in FIG. 9, each round of filtering uses the node set from the last round of filtering as input.

Basic Local Alignment Search Tool (BLAST) is used to find regions of similarity between the nodes and sequences in a given BLAST database. Nodes have previously been filtered to discard nodes without a GEI and short nodes. These nodes are run through BLAST to the probe database to get annotations of the probe sequences as shown in step 118 of FIG. 2.

BLAST is then used to compare these same nodes to the NCBI nucleotide database to get annotations of known nucleotide sequences and identify the host genome (Mizrachi, L (2007). GenBank: the nucleotide sequence database. The AVM handbook [Internet], updated, 22) as shown in step 120 of FIG. 2. Once the nodes are compared to the nucleotide database, the next two rounds of node filtering take place as shown in step 122-1 and 122-2 of FIG. 2. Nodes filtered at this stage are still visualized at a later step in the pipeline, but not annotated by the protein database to reduce CPU usage since protein annotation is significantly slower than other steps in the pipeline. Nodes marked as “Likely Natural” or “Likely Contamination” are filtered and excluded from protein annotation. Due to the initial focus on identifying genetically engineered organisms, GEI that were in their natural location were not of interest and thus filtered out to expend computational time only on those nodes of interest that contained potentially engineered regions. The natural locations are stored in a csv file and were determined by reviewing results of samples that naturally contained the GEI as annotated by subject matter experts. All of the GEI found are checked against the expected organism, start position, and end positions to determine whether they are natural. Any nodes with GEI in natural locations, based on our manually curated list of GEI natural locations, are marked as “likely natural,” see step 122-1 of FIG. 2.

Next, nodes that are not marked as “likely natural” are sent through a contamination filter (see step 122-2 of FIG. 2). Nodes are classified as likely contaminated if the RPKM (reads per kilobase of sequence aligned to per million base pairs sequenced) is lower than a threshold value (log₁₀RPKM>5) which was empirically derived. Nodes that are not contaminated are then compared to the NCBI protein database (see step 122-3 of FIG. 2). These nodes are the candidates for engineering and protein annotations are useful in making the final classification. The annotations allow experts to better understand the type and potential effect of the engineering making protein annotation valuable despite the many CPU hours needed for this step.

The last stage of the pipeline is to summarize and visualize the results in step 124-1. For the original application of identifying genetically engineered organisms, the system produces automated predictions for whether each sample is engineered, but also produces a series of tables and figures that subject matter experts (SMEs) can review to quickly determine whether a sample contains any GEI and what has been uncovered about their surroundings. An important part of this process is the combining and prioritizing of the annotation (BLAST) results. When searching the larger nucleotide and protein databases, upwards of 50 results can be returned—the system combines similar results and selects those that are most distinct and, as a result, provide as much context as possible.

Nodes not marked as likely natural or likely contamination are marked as likely engineered but SMEs review the classifications using the visualizations to make the final call. The database of likely natural GEI is not always comprehensive, and it is important to distinguish between low level cross-over events or poor assembly and intentional engineering. Static and interactive outputs are produced to visualize the read depth and annotations for every node deemed important by filtering.

All of the files used for the visualizations as well as the summary files (see steps 124-1 and 124-2 FIG. 2) are written to a single results folder.

The results folder includes a readme explaining the results files. The results folder also has a summary folder that contains files with information on all the samples. The files include the engineering summary table (engineered caller) which summarizes the signs of engineering found per sample by tabulating which GEI were found, their sizes, read depths, and their suspected natural or engineered status within each sample. A sample is classified as engineered if there is at least one node that contains a GEI and is not natural or contaminated. This file also contains the number of nodes at each filtering stage. The results folder also includes a GEI summary table and a table of reads aligned to each GEI that contains a row for each sample and a column for each GEI and containing the number of reads that aligned to that GEI. The files report raw read counts, reads per million reads sequenced (RPM) and reads per kilobase of engineering marker and million reads sequenced (RPKM). Also included is an alignment stats summary file—containing overall percent aligned of reads to the GEI, number of fully-mapped and half-mapped reads, and percent of reads belonging to each category. This file also displays the number of reads before and after stages in the pipeline such as trimming, downsampling, and peak removal.

The results folder also contains a folder for each sample with more detailed information about each sample and all the figures. Included is a summary file for each sample to describe the contents of each node with data including a list of GEI found, alignment length per GEI, read depth over node, RPKM, and if the node is likely natural. Summary files for scaffolds contain additional information such as the number of unknown base pairs per node. The summary file also contains data on unassembled GEI found in the node (i.e., some reads aligned but the GEI was not found in assembly results). A file merging all BLAST hits for the relevant nodes is created for use during visualization.

During visualization, the data obtained about the engineering is made into visualizations to simplify understanding and interpretation be a SME reviewer. Since hundreds of BLAST hits can be found for a single node, algorithms were designed and implemented to select a subset to include based on the length and quality of the matches as well as maximizing how much of the node is covered by annotation. The BLAST annotations enter the visualization step as one merged data frame, separated by source: GEI, probes, nucleotides, proteins. The visualizations show all data from the GEI and probe annotations since these tend to be limited in number and are the most important for interpretation. The nucleotide and protein annotations, however, are processed through an optimization algorithm to limit the number of results visualized.

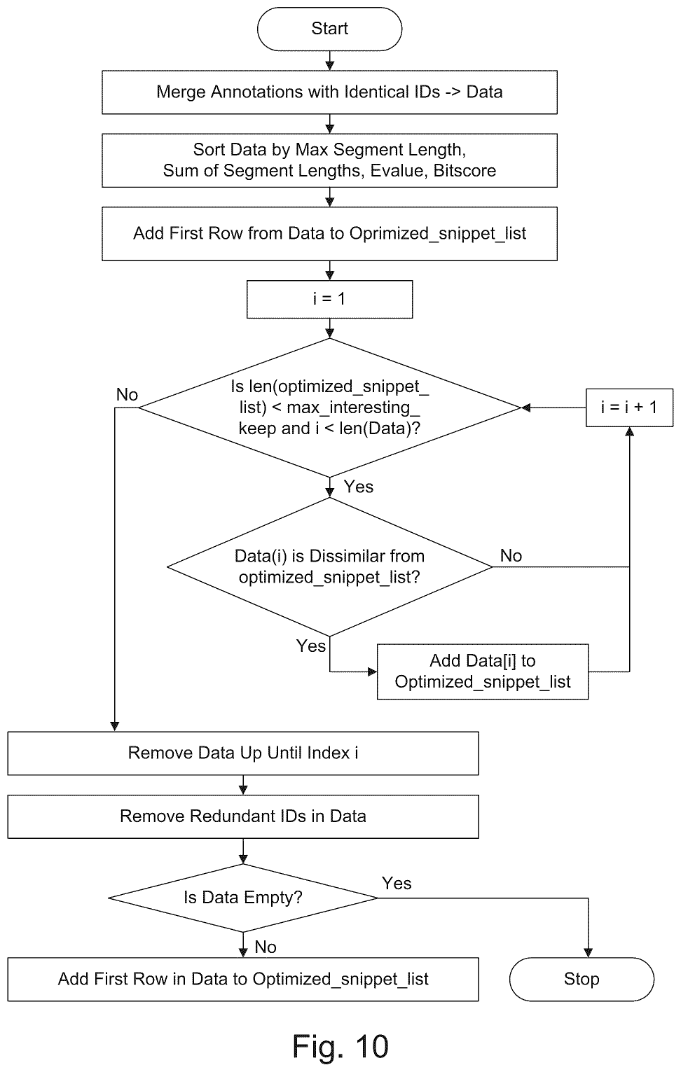

The data (either nucleotide or protein annotations) is first reorganized so that all the results from a single BLAST ID are stored together (as opposed to a new line for each BLAST ID and each match to the node). The data is then sorted by the maximum length of a single segment, with tiebreaks for total length, then evalue, then bitscore. The top few (3 or 5) dissimilar IDs are first included in the visualization. “Similar” IDs are IDs in which there are the same number of segments, the segments each have similar starting and ending points, and the first two words in the name are the same. Additionally, all vectors are considered similar and combined since these results generally are less valuable. A vector is defined as an ID with a name that contains one of the following words: vector, plasmid, synthetic construct, or hypothetical. IDs checked when searching for dissimilar IDs and redundant IDs are then removed from the rest of the data. A redundant ID is one that does not cover any new base pairs in the node (within 20 base pairs from already covered regions). The top ID of this filtered data is then included in the visualization. This process of removing redundant IDs then selecting the top remaining ID is performed until there are no non-redundant IDs left. The final IDs are shown in both the static and dynamic visualizations. This algorithm is shown in FIG. 10 which is a flowchart for BLAST optimization algorithm. “Optimized snippet list” refers to the IDs that are included in the final visualizations. “Data” refers to the initial data frame with all IDs. “Max interesting keep” is the number of dissimilar IDs to pick initially, before optimizing for non-redundant coverage.

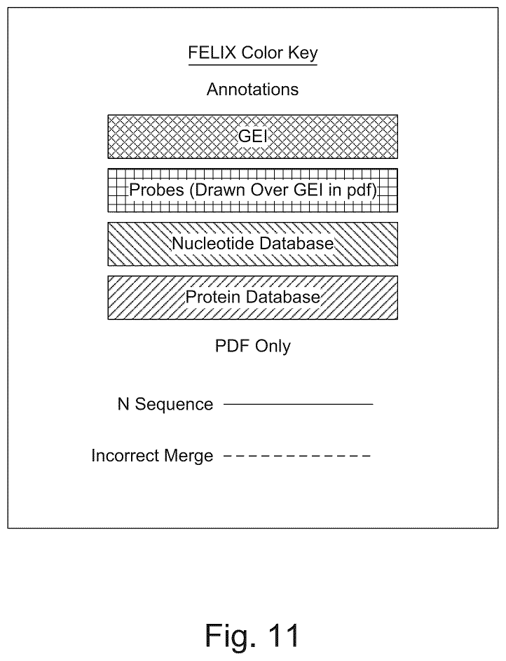

The static visualizations are saved as pdfs making them near instantaneous to load and very useful for quick review of a sample. The notation scheme is used for both sets of visualizations are shown in FIG. 11, which is Key for the subsequent IGV screenshots.

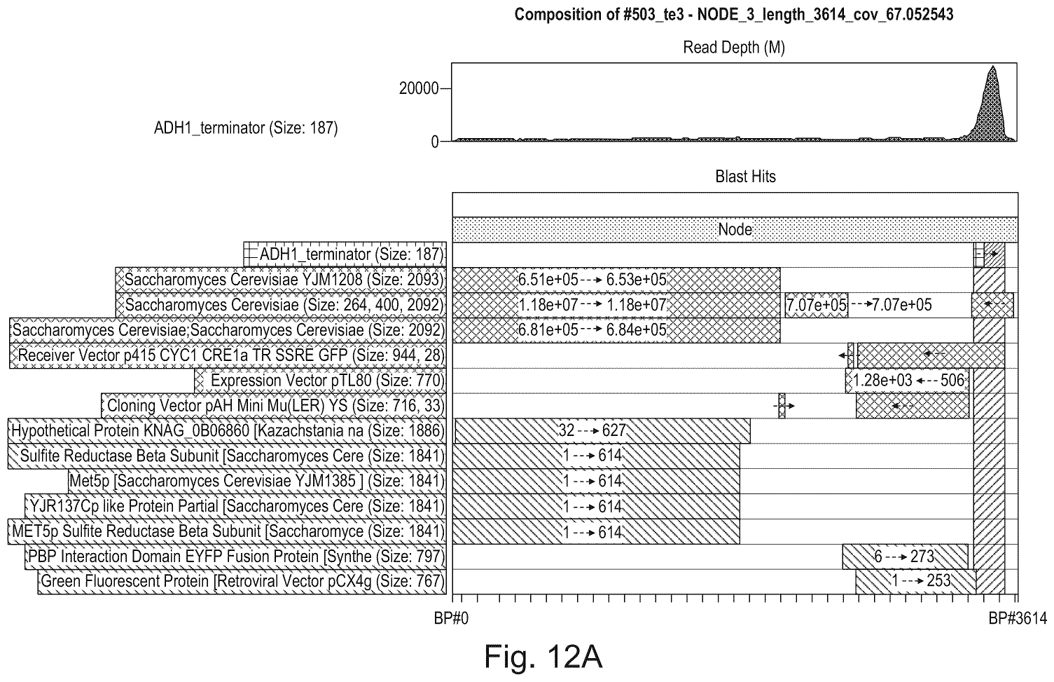

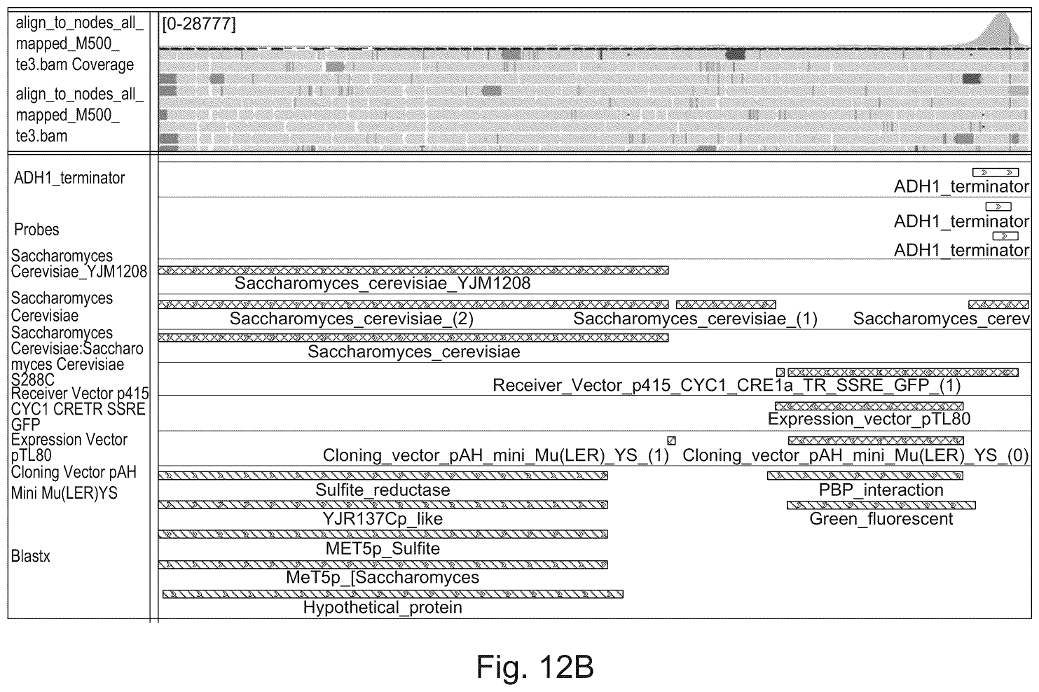

FIG. 12A is a screenshot of a static and FIG. 12B is screenshot of dynamic visualizations of engineering in a sample as part of the GUI 87 displayed on the display 84. Specifically, the ADH1 terminator is shown on one edge with over 3 kb of surrounding information that shows it is near GFP (bottom bar) inserted into S. cerevisiae (top bar) at the Met5p locus (3rd from bottom bar).

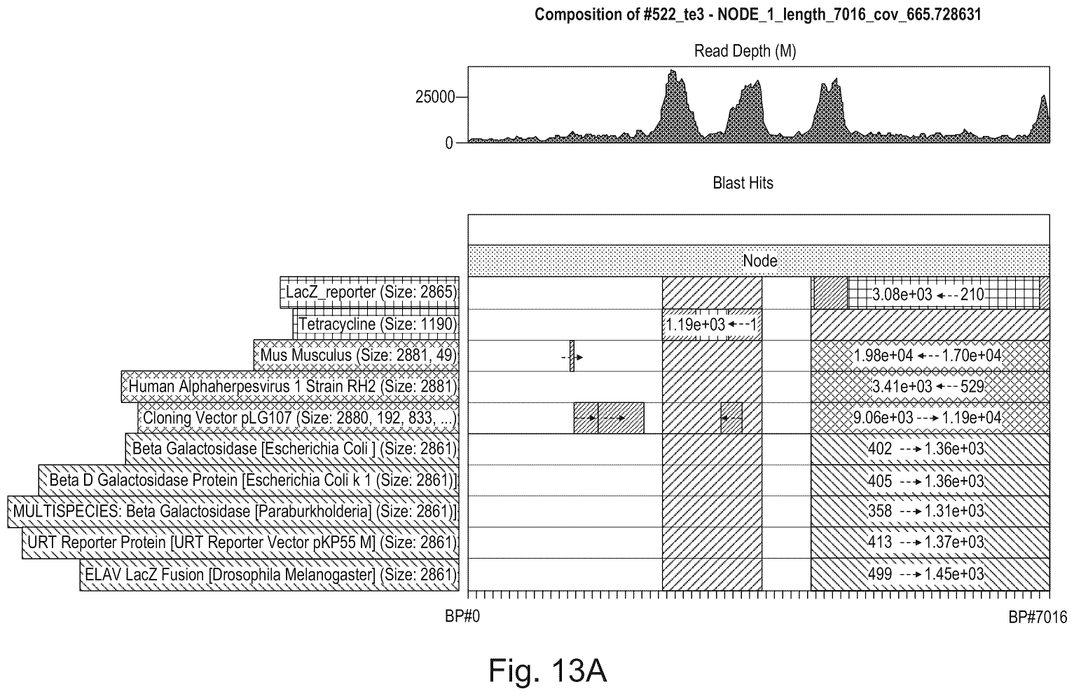

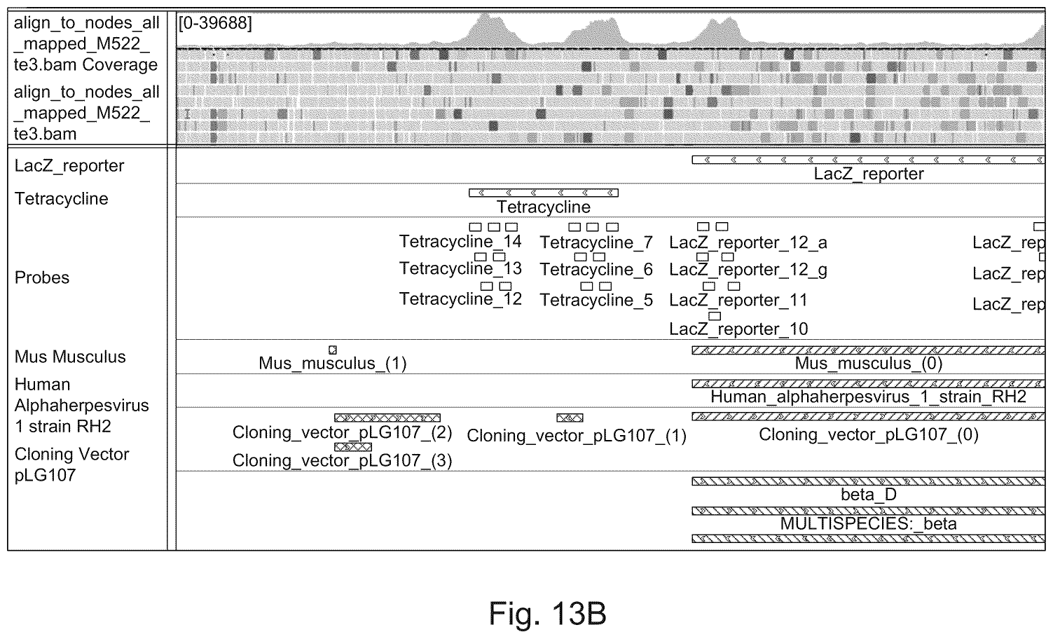

FIG. 13A is a screenshot of a static and FIG. 13B is a screenshot of dynamic visualizations of engineering in a sample displayed as part of the GUI 87 displayed on the display 84. This node shows lacZ and the tetracycline resistance determinant. Additional analysis of other nodes in the sample showed that this is part of a 6 kb insertion into P. aeruginosa.

As shown in FIGS. 12A and 12B and 13A and 13B, along the top, are a histogram of the read depth along the node. Below that the figures show the BLAST annotations coded by type (organism/nucleotide, protein, probes, and GEI).

In cases where assembly was not perfect, the engineered region can be spread across multiple nodes, often each with one or a few GEI. When this happens, looking at the results in more detail through an interactive visualization can be useful. For interactive visualizations, see the Broad Institute's Integrative Genomics Viewer (IGV) (Thorvaldsdóttir, H., Robinson, J. T., & Mesirov, J. P. (2013). Integrative Genomics Viewer (IGV): high-performance genomics data visualization and exploration. Briefings in bioinformatics, 14(2). 178-192). This interactive platform allows a user to zoom in on any region and analyze the read alignment in more detail, down to the nucleotide level. FIG. 13B shows an example of this through a contig that contains only part of the engineered region. Some of the small rectangles between the histogram and BLAST results represent individual reads whose other half is aligned to a different node that contains a small portion of lacZ and the integration point in the P. aeruginosa genome. Because of this split of engineering markers across multiple contigs, the sample was reanalyzed using scaffolds which connected the two contigs into a single node.

These IGV figures are loaded using a batch script produced by the pipeline that tells IGV where the alignment and annotation data is stored (See step 124-4 of FIG. 2). The read depths are loaded in from BAM files. GFF files are loaded to view BLAST annotations.

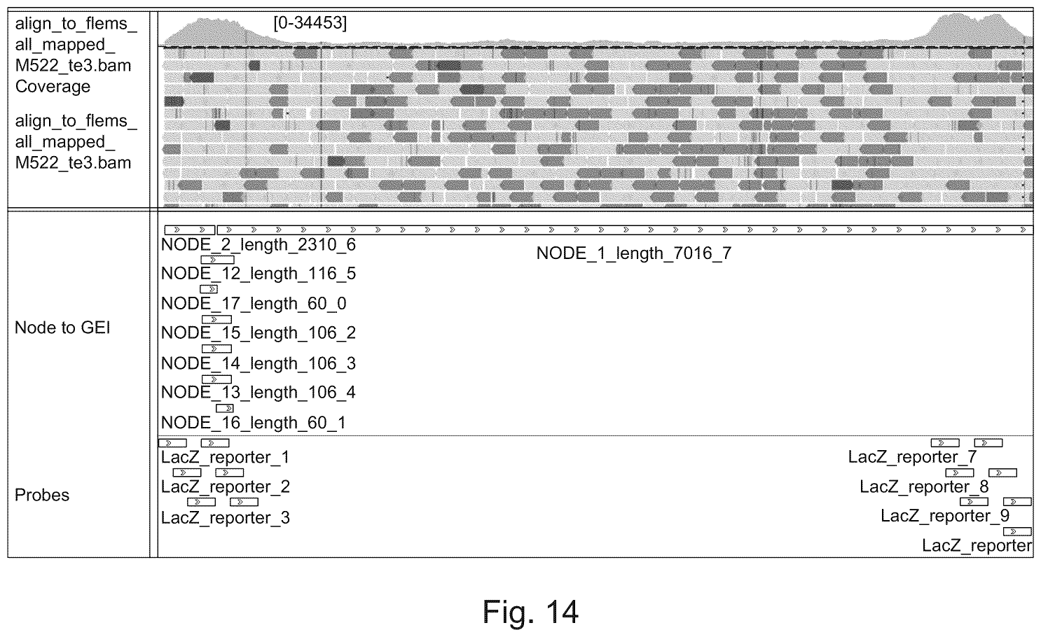

In addition to showing the annotations on the nodes, IGV also allows viewing of the nodes on the GEI. Specifically, FIG. 14 shows the alignment of reads and contigs to lacZ for T&E sample T299. This shows that lacZ was not assembled into a single contig but instead split across nodes 1 and 2 with smaller portions showing up in some smaller nodes. Every GEI found in the sample (assembled or not) is available to view. This is particularly useful for the rare case in which a GEI is not assembled but has a few hundred reads or more that align to it. In that case, generally the reads all align to the same region and viewing the GEI in IGV allows one to distinguish between that and some other unexpected assembly failure.

The pipeline uses a yaml-formatted configuration (“config”) file that can be altered by the user to define various parameters and enable outputs. The features explained in this additional analysis section can be enabled and configured through the config file.

The pipeline has the feature of aligning all samples to given genomes or sequences. A list of sequence names can be provided in the config section for “extra alignments” or “extra genomes”. A FASTA file must be provided for each sequence. Result files per alignment include an alignment summary spreadsheet, reads/RPM/RPKM per chromosome, and a batch file to view all alignments in IGV. For the sequences listed under “extra genomes”, an extra output of a heatmap is produced. Samples are sorted into categories based on which genome had the most mapped reads.

Samples can be grouped and aligned to the best assembled sample in the group. To group samples, the user lists the sample names in the relevant section of the config file. The assembly quality algorithm is used to determine which sample has the best assembly. All samples are then aligned to this best sample and the results are displayed as an array of histograms over the node of the best sample. This feature can be utilized to test the results of an experiment, for example when different wash conditions are used on the same genetic material.

A crosstalk metric has been created to predict whether a GEI exists in a sample from crosstalk or not. Crosstalk can occur between samples enriched in the same pool if an error occurs during the enrichment/PCR/sequencing process causing the adaptors from one sample to be attached to a sequence that originated from a different sample. This generally is rare and occurs at low levels. Therefore, the system uses developed a metric to identify when this has occurred to prevent errors in the final calls. A score is assigned to every GEI in a sample and a higher score means the GEI is more likely to be the results of crosstalk/contamination. The score is a weighted sum from various sub-metrics that represent different patterns that have been observed to be indicative of crosstalk. Every GEI receives a score relative to other instances of the same GEI from other samples. The GEI in the other samples is referred to as the ‘donor’ whereas the GEI being considered is the ‘recipient’. Only the highest score per recipient GEI is considered when determining contamination as this represents the most likely donor or source for the crosstalk.

There are multiple factors that go into the score, relating to the donor GEI and sample, recipient GEI and sample, and relation between the donor and recipient. These metrics aim to quantify the factors that an expert uses to make the classification of crosstalk real.

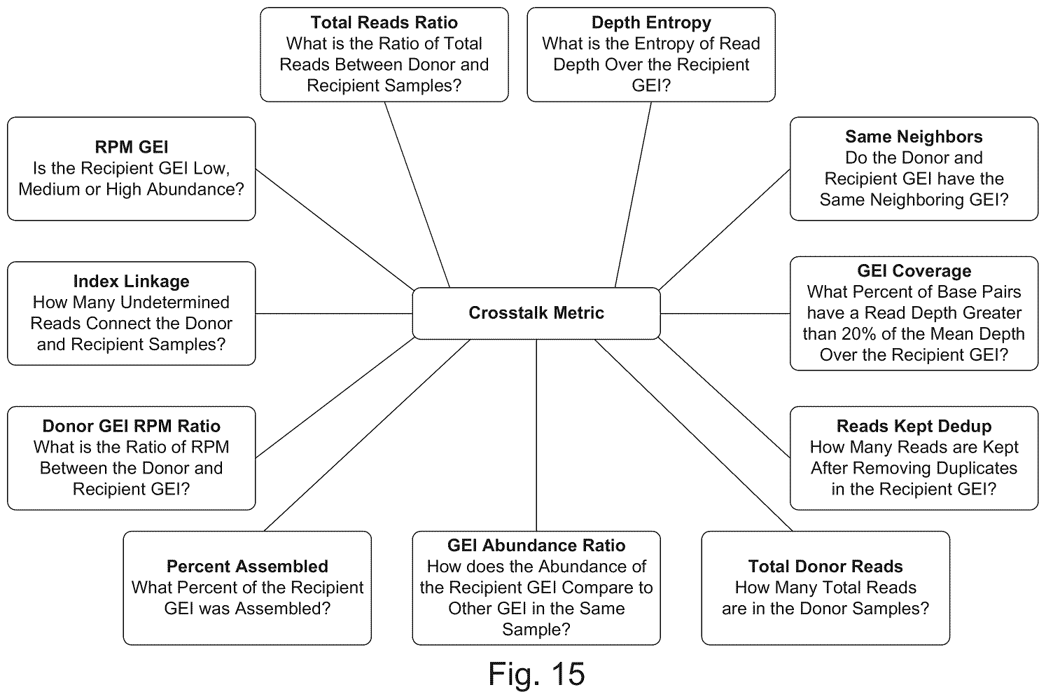

FIG. 15 depicts the questions asked by experts that have been quantified by the metric.

In general, if the donor GEI is high abundance, the recipient GEI is low abundance, and there are undetermined reads spanning the two samples, the system then assesses that it is likely to believe crosstalk occurred and be able to specify the source. Undetermined reads refer to reads that were not used in the analysis because they did not have one of the expected i5/i7 barcode pairs, and instead had barcodes for two different samples.

Prior to running the pipeline, reads are demultiplexed, or separated into individual sample files, based upon their i5/i7 barcodes which are short sequences at the ends of each read designed for this purpose. In a small number of cases, the barcodes at the two ends of a read will not correspond to the same sample in which case, it is classified as an undetermined read and put in a separate file. It has been found that the number of undetermined reads whose indices correspond to two samples correlates to the amount of crosstalk between the samples. Samples where more single crossover events have occurred are also more likely to have had double crossover events that would lead to a read being classified as the wrong sample and therefore being crosstalk/contamination.

The pipeline is run through a custom-made python wrapper rather than run through the snakemake command directly. A variety of features and automations have been implemented in the wrapper such as notification setup, a timer, SLURM batch script creation, and multi-config file merging. These features are described in more detail below.

The pipeline has a feature of notifying a user upon certain events. The notification takes place over email from an email account made for this purpose. An argument can be passed when running the wrapper that will setup email notifications. Notifications will be sent upon starting the pipeline (if submitted via SLURM), when an error occurs, and when the pipeline finishes. This makes monitoring the analysis progress simpler.

The wrapper runs the snakemake pipeline through a timer when the pipeline is being run on a cluster that limits the node usage. Prior to setting the timer, snakemake would be stopped abruptly when the user's time on the node ran out. By implementing the timer, the snakemake pipeline can be stopped cleanly before the time on the node runs out and the pipeline will therefore be unlocked for the next use.

The wrapper automates the process of creating a batch script necessary to run the pipeline using the SLURM scheduler on a cluster. The wrapper will ensure all resource requests are valid so the user does not end up submitting an invalid resource request. The wrapper will automatically submit this batch script.

One yaml file can be used as a configfile for snakemake. The wrapper merges multiple config files as specified by the user and changes parameters as specified by command line arguments to the wrapper. This allows for files to be created and saved for custom runs without having to change the main config file.

While this invention has been particularly shown and described with references to preferred embodiments thereof, it will be understood by those skilled in the art that various changes in form and details may be made therein without departing from the scope of the invention encompassed by the appended claims.

What is claimed is:

1. A system, comprising:
a DNA sequencer that generates a series of reads associated with a sample containing DNA; and
a computing device that analyzes the reads to assemble, annotate, and/or visualize genetic elements of interest.

2. The system of claim 1, wherein the DNA sequencer is used in combination with probe based enrichment.

3. The system of claim 1, wherein the computing device employs smart-filtering to select reads for assembly that will optimize assembly output regardless of the number or distribution of reads.

4. The system of claim 3, wherein the reads are selected based on how much information they contain about the genetic elements of interest and its flanking region.

5. The system of claim 3, wherein the reads are broken into the three types: fully mapped, half mapped, and unmapped reads.

6. The system of claim 3, wherein the computing device performs assembly using many half-mapped reads, a smaller number of fully mapped reads, and the smallest number of unmapped reads.

7. The system of claim 1, wherein the computing device employs an assembly quality analysis to determine which node sets are to be used.

8. The system of claim 1, wherein the computing device employs smart selector.

9. The system of claim 1, wherein the computing device employs an assembly quality score that is used to compare different runs of the same sample in the sequencer to score and rank for each run.

10. The system of claim 9, wherein the runs are compared using the average ranking, and the run with the lowest average ranking was set as the default for the pipeline.

11. The system of claim 1, wherein the computing device employs a crosstalk metric created to predict whether a GEI exists in a sample from crosstalk or not.

12. The system of claim 1, wherein the system is a laboratory instrument.

13. The system of claim 1, wherein the system is a field instrument to analyze waste water, for example.

14. A method of analysis of a DNA-containing sample, the method comprising:
generating a series of reads associated with a sample containing DNA using a sequencer; and
analyzing the reads to assemble, annotate, and/or visualize genetic elements of interest.

15. The method of claim 14, further comprising employing smart-filtering to select reads for assembly that will optimize assembly output regardless of the number or distribution of reads.

16. The method of claim 15, wherein the reads are selected based on how much information they contain about the genetic elements of interest and its flanking region.

17. The method of claim 14, further comprising employing an assembly quality analysis to determine which node sets are to be used.

18. The method of claim 14, further comprising employing smart selection.

19. The method of claim 14, further comprising employing an assembly quality score that is used to compare different runs of the same sample in the sequencer to score and rank for each run.

20. The method of claim 14, further comprising employing a crosstalk metric to predict whether a GEI exists in a sample from crosstalk or not.
